(19) United States
(12) Plant Patent Application Publication
(10) Pub. No.: US 2002/0148019 P1
(43) Pub. Date: Oct. 10, 2002

(54) Plumbago auriculata named 'Monite'

(76) Inventor: Brad Alan Crocker, Aliseo Viejo, CA (US)

Correspondence Address: Brad Alan Crocker, Botanical Department, Monrovia Nursery, 18331 E. Foothill Blvd., Azusa, CA, 91702, US

(21) Appl. No.: 09/826,222

(22) Filed: Apr. 5, 2001

(51) Int. Cl. A01H 5/00

(52) U.S. Cl. PLT/226

(57) ABSTRACT

A new and distinct selection of Plumbago shrub, denominated as Plumbago auriculata named ‘Monite’, substantially as shown and described.

### BACKGROUND

This invention relates to a new and distinct selection of Plumbago auriculata, which is native to Southern Africa, and a member of the Leadwort family. The new variety, Plumbago auriculata cv. ‘Monite’ resulted from a naturally occurring, whole plant mutation which was discovered in May of 1999 in a cultivated planting of Plumbago auriculata ‘Monott’ (U.S. Plant Pat. #7822). ‘Monite’ exhibited a white flower in #1 nursery containers in Azusa, Calif.. The ‘Monite’ selection differs from ‘Monott’ by possessing a white flower versus the rich blue flower of ‘Monott’. ‘Monite’ differs from other Plumbago plants by its combination of floriferous habit of white flowers and larger and wider petals and a more dense compact growth habit.

The new plant has been asexually reproduced by cuttings since its discovery in 1999 in Azusa, Calif.. Through extensive asexual propagation of this plant and observation, it has been established that the plant is true to type and the novel, exceptional characteristics of this new plant are fixed and stable and reliably passed on to progeny through asexual reproduction.

### SUMMARY

Plumbago auriculata cultivar ‘Monite’ possesses a white flower. Other white flowering Plumbago plants do exist, however; the individual flowers do not exhibit the larger size and fullness associated with the overlapping flower petals of cultivar ‘Monite’.

The compact growth habit of cultivar ‘Monite’ sets this plant apart from other Plumbago plants which are generally sprawling, mounding plants with a height of 6-8 feet and 8-10 foot spread. The growth habit of cultivar ‘Monite’ is slightly smaller than ‘Monott’.

The cultivar ‘Nonite’ typically will bloom for eight to nine months of the year in Southern California conunencing bloom before even the early cultivar ‘Monott’. Comparison of other Plumbago plants demonstrate cultivar ‘Monite’ has the propensity to conspicuously grow less and flower more when grown under similar conditions.

### BRIEF DESCRIPTION OF THE DRAWING

FIG. 1 photograph exhibits a close up of the inflorescence of cultivar ‘Monite’.

FIG. 2 photograph shows the comparison of cultivar ‘Monite’ above and cultivar ‘Monott’ below and the similarity of flower size and contrast of flower color.

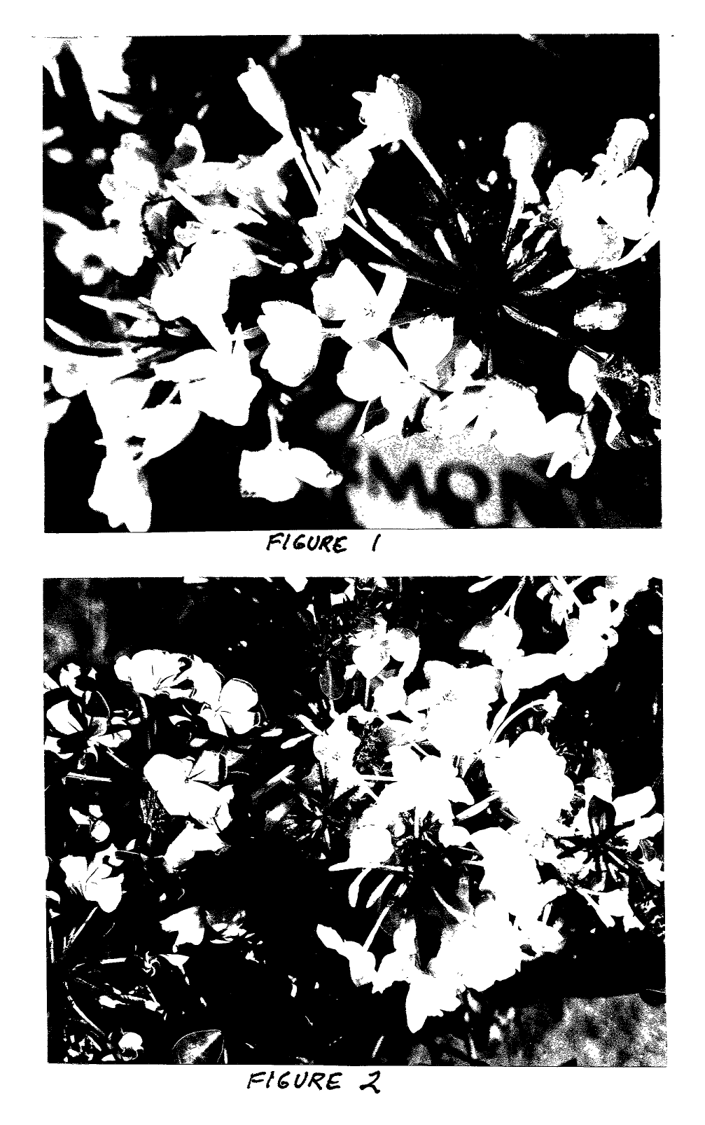

### BOTANICAL DESCRIPTION

Below is a detailed description of Plumbago auriculata cultivar ‘Monite’. Color terminology is from the Royal Horticultural Society Colour Charts. Measurements are based on plants grown in Southern California.

Overall habit: mounding shrub

Overall size: Maximum height from soil surface to top of foliage ranges from 120 cm to 150 cm Maximum width of the plant ranges from 120 cm to 150 cm

Flowering time in Southern California: Flowering commences in early March and continues until November.

FLOWER:

Arrangement.—terminal, spike like racemes

Corolla diameter.—averaging 2.5 to 3 cm in width

Corolla color.—White Group 155D

Corolla.—tubular with 5 parted lobes

tube.—typically 3 cm in length; 2 mm in width lobes: slightly overlapping, obovate in shape, acute base averaging 1.5 cm long and 1 cm wide, prominent midrib, slightly depressed. texture: thin, glabrous

Calyx.—tubular, 5 parted at tip color: Green Group 141C size: averaging 1.3 cm in length, 3 mm in width surface: upper half of calyxwith stalked glands, Greyed-Red Group 180C bracts: 3 at base, 6-7 mm long and 2 mm wide, color Green Group 141C Stamens: 5 in number, adhering to within throat arising above corolla tube. anthers: length averaging 2 mm width: less than 1 mm color: Violet-Blue 92B filaments: length averaging 3 cm width: less than 1 mm, very slender color: White Group 155D Pistil: averaging 2 cm in length. Color: White Group 155D

FRUIT: to date not observed.

FOLIAGE:

Size.—Length: ranges from 3.5 cm to 6.5 cm Width: ranges from 1 cm to 2 cm

Shape.—Overall: oblong to oblong—spatulate to obtuse Base: auricled, wrapping around stem, narrowly acute. Tip: shortly mucronate.

Margin.—entire, slightly undulate

Color.—mature upper surface: Yellow Green Group 146A mature lower surface: Yellow Green Group 146B juvenile upper surface: Yellow Green Group 146B juvenile lower surface: Yellow Green Group 146C

Texture.—thin, glabrous

Arrangement.—alternate

BRANCHLETS:

juvenile.—lightly ribbed, 2-3 mm in diameter color: Yellow Green Group 146B

mature.—3 mm and larger in diameter color: Red-Purple Group 59C mixed with Yellow Green Group 146B

DISEASE RESISTANCE: As is typical of this species, ‘Monite’ cultivar exhibited a strong resistance to disease under normal growing conditions.

I claim:

1. A new and distinct Plumbago auriculata plant named ‘Monite’ substantially as shown and described which differs distinctly from other Plumbago auriculata plants by its unique combination of longer bloom season, of floriferous habit of white flowers with larger and wider petals and a more dense compact growth habit.
